(19) United States
(12) Patent Application Publication
(10) Pub. No.: US 2005/0203163 A1
(43) Pub. Date: Sep. 15, 2005

(54) Farnesyl transferase inhibitors in combination with HMG CoA reductase inhibitors for the treatment of cancer

(75) Inventor: Shama Mohammed Kajiji, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/004,635

(22) Filed: Dec. 3, 2004

Related U.S. Application Data
(63) Continuation of application No. 10/217,108, filed on Aug. 12, 2002, now abandoned.
Application No. 10/217,108 is a continuation of application No. 09/367,435, filed on Oct. 25, 1999, now abandoned.
(60) Provisional application No. 60/049,638, filed on Jun. 16, 1997.

(51) Int. Cl. A61K 31/405; A61K 31/4015; A61K 31/401; A61K 31/366; A61K 31/365; A61K 31/225

(52) U.S. Cl. 514/414; 514/468; 514/424; 514/423; 514/460; 514/548

(57) ABSTRACT

The present invention relates to a method of treating cancer in a mammal, including a human, by administering to the mammal a FTase inhibitor in combination with an HMG CoA reductase inhibitor.

This application is a continuation application of U.S. Ser. No. 09/367,435, filed Oct. 25, 1999, which is a 35 U.S.C. §371 application of PCT/IB98/00881, filed Jun. 5, 1998 which claims priority to U.S. Provisional Patent Application Ser. No. 60/049,638, filed Jun. 16, 1998.

This invention relates to the use of a farnesyl transferase (FTase) inhibitor in combination with a hydroxymethylglutaryl coenzyme A (HMG CoA) reductase inhibitor to treat cancer in a mammal.

Oncogenes are genes that, when activated, encode protein components of signal transduction pathways which lead to the abnormal stimulation of cell growth and mitogenesis. Oncogene expression in cultured cells leads to cellular transformation, characterized by the ability of cells to grow in soft agar and the growth of cells as dense foci lacking the contact inhibition exhibited by non-transformed cells.

Mutation and/or overexpression of certain oncogenes is frequently associated with human cancers and other disorders involving abnormal (i.e., unregulated) cell growth. For example, the growth of benign and malignant tumors can be caused by the expression of an activated Ras oncogene or by activation of the Ras protein by another gene that has undergone oncogenic mutation. The abnormal growth of cells that occurs in the benign and malignant cells of other proliferative disorders can be caused by aberrant Ras activation. Mutated, oncogenic forms of Ras are frequently found in many human cancers, most notably in more than 50% of colon and pancreatic carcinomas (Kohl et al., Science, Vol. 260, 1834 to 1837, 1993). The Ras oncogene is expressed in about 40% of solid malignant tumors that are unresponsive to conventional chemotherapies. The K-Ras isoform is expressed in about 90% of pancreatic tumors and about 40% of colorectal and lung cancers. The H-Ras isoform is expressed in about 40% of head and neck cancers. The N-Ras isoform is expressed in most thyroid cancers and about 25% of acute myeloid leukemias. To acquire the potential to transform normal cells into cancer cells or benign cells that exhibit abnormal growth, as defined below, the precursor of the Ras oncoprotein must undergo farnesylation of the cysteine residue located in a carboxy-terminal tetrapeptide. Inhibitors of the enzyme that catalyzes this modification, farnesyl protein transferase, are therefore useful as anticancer agents for tumors in which Ras contributes to transformation.

The K-Ras isoform can be both farnesylated and geranyl-geranylated in intact cells. Potent inhibitors of the enzyme farnesyl (FTase) that are highly selective for FTase versus geranylgeranyl transferase I (GGTase I) can be incapable of blocking prenylation of mutant K-Ras and therefore ineffective at inhibiting growth of K-Ras expressing tumor cells.

The present inventor has found that the administration of a low dose HMG CoA reductase inhibitor in combination with a potent selective FTase inhibitor will block K-Ras prenylation and K-Ras function, as well as H-Ras prenylation and function. The activity of the protein prenyl transferases FTase and GGTase I is dependent on the concentrations of the isoprenoid substrates, farnesyl- and geranylgeranyl-pyrophosphates, respectively. Mevalonate is the first committed intermediate in the isoprenoid pathway, and its synthesis is dependent on the activity of HMG CoA reductase. Compounds such as lovastatin and compactin, which are tight binding inhibitors of HMG CoA reductase, block mevalonate formation and thus block the isoprenoid pathway. They therefore inhibit both FTase and GGTase I.

The therapeutic effect of compounds from the two above classes of drugs (FTase inhibitor and HMG CoA reductase inhibitor) is believed to be synergistic. The present inventor has found that the combined administration of an FTase inhibitor and an HMG CoA reductase inhibitor overcomes the limitations of each given separately. The combination is therefore expected to be effective in cases where either agent alone would not be effective.

Japanese Patent Application JP7316076A, which was published on Dec. 5, 1995, refers to an anticancer pharmaceutical composition that contains limonene, which, while not a FTase inhibitor, has been shown to impair the incorporation of mevalonic acid-derived isoprene compounds into Ras and Ras related proteins, and pravastatin, which is an HMG CoA reductase inhibitor.

The present invention relates to a pharmaceutical composition for the treatment of cancer or a benign proliferative disorder in a mammal, including a human, comprising a FTase inhibitor, an HMG CoA reductase inhibitor and a pharmaceutically acceptable carrier, wherein the active ingredients in such composition (i.e., the FTase inhibitor and the HMG CoA reductase inhibitor) are present in amounts that render the composition effective in the treatment of cancer or a benign proliferative disorder.

This invention also relates to a method of treating cancer or a benign proliferative disorder in a mammal, including a human, comprising administering to said mammal an anticancer or antiproliferative effective amount of a pharmaceutical composition comprising a FTase inhibitor, an HMG CoA reductase inhibitor and a pharmaceutically acceptable carrier.

This invention also relates to a method of treating cancer or a benign proliferative disorder in a mammal, including a human, comprising administering to said mammal a FTase inhibitor and an HMG CoA reductase inhibitor in amounts that render the combination of such two active agents effective in the treatment of cancer or a benign proliferative disorder.

This invention also relates to a pharmaceutical composition for inhibiting the abnormal growth of cells in a mammal, including a human, comprising a FTase inhibitor, an HMG CoA reductase inhibitor and a pharmaceutically acceptable carrier, wherein the active ingredients in such composition (i.e., the FTase inhibitor and the HMG CoA reductase inhibitor) are present in amounts that render the composition effective in inhibiting the abnormal growth of cells.

This invention also relates to a method of inhibiting the abnormal growth of cells in a mammal, including a human, comprising administering to said mammal a FTase inhibitor and an HMG CoA reductase inhibitor in amounts that render the combination of such two active ingredients effective in inhibiting the abnormal growth of cells.

The term “treating, as used herein, refers to preventing, or retarding or inhibiting the progress of the disorder to which such term is applied.

“Abnormal cell growth”, as used herein, refers to cell growth that is independent of normal regulatory mechanisms (e.g., loss of contact inhibition). This includes the abnormal growth of: (1) tumor cells (tumors) expressing an activated Ras oncogene; (2) tumor cells in which the Ras protein is activated as a result of oncogenic mutation in another gene; and (3) benign and malignant cells of other proliferative diseases in which aberrant Ras activation occurs.

Examples of such benign proliferative diseases are psoriasis, benign prostatic hypertrophy and restenosis.

Patients that can be treated with a FTase inhibitor in combination with an HMG CoA reductase inhibitor according to the methods of this invention or using the pharmaceutical compositions of the invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphonas, cancer of the bladder, cancer of the kidney or ureter (e.g., renal cell carcinoma, carcinoma of the renal pelvis), or neoplasms of the central nervous system (e.g., primary CNS lymphona, spinal axis tumors, brain stem gliomas or pituitary adenomas).

Patients that can be treated with a FTase inhibitor in combination with an HMG CoA reduction inhibitor according to the methods of this invention or using the pharmaceutical compositions of the invention also include patients suffering from abnormal cell growth, as defined above.

More specific embodiments of this invention relate to the above pharmaceutical compositions and methods of treatment wherein the FTase inhibitor is selected from:
(a) compounds of the formula 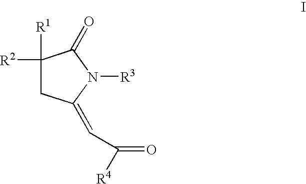
wherein R¹ and R² are independently selected from the group consisting of —(CH₂)ₚ(5-10 membered heterocycles), —(CH₂)ₚ(C₆-C₁₀ aryl), allyl, propargyl and C₁-C₆ alkyl wherein p is 0 to 3, said alkyl and the alkyl moieties of said R¹ and R² groups are optionally substituted by 1 to 3 R⁹ substituents, and the aryl and heterocyclic moieties of said R¹ and R² groups are optionally substituted by 1 to 3 substituents independently selected from halo and R⁹;
R³ is —(CH₂)ₘ(1- or 2-adamantyl), —(CH₂)ₘ(C₃-C₁₀ cycloalkyl), —(CH₂)ₘ(C₆-C₁₀ aryl), C₁C₁₀ alkyl, 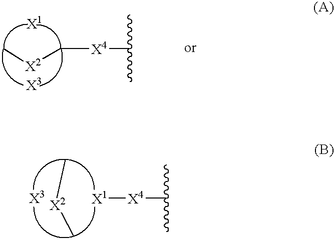
wherein m is 0 to 6, and said cycloalkyl and alkyl optionally contain I or 2 double or triple bonds;
X¹, X², and X³ are each independently C₁-C₇ alkylene optionally containing 1 or 2 double or triple bonds, X⁴ is a bond or C₁-C₇ alkylene optionally containing 1 or 2 double or triple bonds, and, in formula (B), the X⁴ moiety is attached to the X¹ moiety at any available carbon in the X¹ moiety;
R⁴ is C₆-C₁₀ aryl, 5-10 membered heterocyclyl or C₁-C₆ alkyl wherein each of said R⁴ groups is optionally substituted by 1 to 3 R⁵ substituents;
each R⁵ is independently selected from the group consisting of halo, nitro, cyano, phenyl, —C(O)OR⁶, —SO₂NR⁶R⁷, —NR⁶R⁸, —C(O)R⁶, —OR⁶, —C(O)NR⁶R⁸, —OC(O)NR⁶R⁸, —NR⁸C(O)NR⁸R⁶, —NR⁸C(O)R⁶, —NR⁸C(O)O(C₁-C₄ alkyl), —C(NR⁸)NR⁸R⁶, —C(NCN)NR⁸R⁶, —C(NCN)S(C₁-C₄ alkyl, —NR⁸C(NCN)S(C₁-C₄ alkyl), —NR⁸C(NCN)NR⁸R⁶, —NR⁸SO₂(C₁-C₄ alkyl), —S(O)ₙ(C₁-C₄ alkyl) wherein n is 0 to 2, —NR⁸C(O)C(O)NR⁸R⁶, —NR⁸C(O)C(O)R⁸, thiazolyl, imidazolyl, oxazolyl, pyrazolyl, triazoyl tetrazolyl, and C₁-C₄ alkyl optionally substituted by 1 to 3 fluoro substituents;
each R⁶ and R⁷ is independently hydrogen or C₁-C₄ alkyl;
each R⁸ is independently R⁵ or —OR⁶; and,
each R⁹ is independently selected from cyano, R⁶, —OR⁶, —OC(O)R⁶, —C(O)OR⁶, —C(O)NR⁶R⁷, —NR⁶R⁷, —NR⁶R⁸, —SO₂NR⁶R⁷, and C₁-C₄ alkyl substituted by hydroxy; and
(b) compounds of the formula 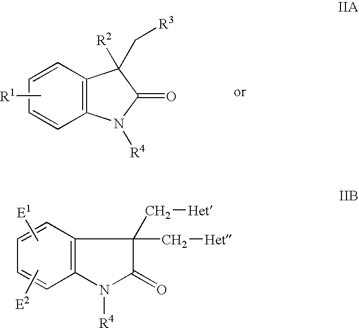
wherein R¹ is hydrogen, halo (e.g., chloro, fluoro, bromo or iodo), cyano, hydroxy, nitro, trifluoromethyl, —NHR⁵, —NR⁵R⁵, R⁵, —OR⁵ or —S(O)ₘ—R⁵;
R² is —(CH₂)ₙ—Y or —OCOR⁵;
R³ is 4-, 3-, or 2-pyridyl, pyrimidyl, pyrazinyl, 2-fluoro-4-pyridyl or 3-fluoro-4-pyridyl;
R⁴ is 1-adamantyl or 2-adamantyl;
Y is hydrogen, hydroxy, amino, cyano, —NHR⁵, —NR⁵R⁵, —NHCOR⁵, —NHCO₂R⁵, halo, OR⁵, —S(O)ₘR⁵, —CO₂H, —CO₂R⁵, —CONR⁵R⁵, —CONHR⁵, —CONH₂, —COR⁵, —CH═CHCO₂R⁵, —OCOR⁵, phenyl, phenyl substituted with W, —C≡CCO₂R⁵, —CH═CHR⁵ or —C≡CR⁵;
each R⁵ is, independently, (C₁-C₄) straight or branched alkyl, phenyl or benzyl, wherein said phenyl and the phenyl moiety of said benzyl may optionally be substituted with halo, hydroxy, nitro, cyano, amino, (C₁-C₄) straight or branched alkyl, (C₁-C₄) straight or branched alkoxy, phenyl, benzyl, (C₁-C₄)alkylamino, di[(C₁-C₄)alkyl]amino, or —S(O)ₘ—(C₁-C₄) straight or branched alkyl;
each W is, independently, halo, R⁵, hydroxy, —OR⁵, nitro, amino, —NHR⁵, —NR⁵R⁵, cyano, or —S(O)ₘ—R⁵;
m is0, 1 or2;
n is 1 to 7;
p is 0 or 1;
E¹ and E² are selected, independently, from hydrogen, halo, (C₁-C₃)alkyl, hydroxy, (C₁-C₃)alkoxy, nitro, trifluoromethyl, cyano, amino, (C₁-C₃)alkylamino and di[(C₁-C₃)alkyl]amino;
and their pharmaceutically acceptable salts.

Het′ and Het″ are selected, independently, from 6 membered heterocyclic rings containing from one to four nitrogen atoms as part of the ring, optionally substituted with one substituent selected from (C₁-C₃)alkyl, halo, hydroxy, (C₁-C₃)alkoxy, amino, (C₁-C₃)alkylamino and di[(C₁-C₃)alkyl]amino; and
(c) compounds of the formula 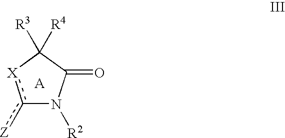
wherein both dotted lines represent optional double bonds;
Z is oxygen or sulfur when it is double bonded to ring A and Z is hydroxy, (C₁-C₁₀)alkyl-S—, (C₁-C₁₀)alkyl-SO—, (C₁-C₁₀)alkyl-SO₂—, adamant-2-yl-S—, naphthyl-S—, benzyl-S —, phenyl-C(═O)CH₂—S—, (C₁-C₆)alkyl-O—C(═O)—CH₂—S— or (H,H) (i.e., Z represents two hydrogen atoms, each of which is single bonded to the same carbon of ring A) when Z is single bonded to ring A, and wherein said naphthyl and phenyl and the phenyl moiety of said benzyl may optionally be substituted with from one to three substituents independently selected from (C₁-C₆)alkyl optionally substituted with from one to three fluorine atoms, (C₁-C₆)alkoxy optionally substituted with from one to three fluorine atoms, halo (e.g., chloro, fluoro, bromo or iodo), amino, (C₁-C₆)alkylamino, [di-(C₁-C₆)alkyl]amino, cyano, nitro, (C₁-C₆)alkyl-SOₙ— wherein n is zero, one or two, —COOH, —COO(C₁-C₆)alkyl and —C(O)NH(C₁-C₆)alkyl;
X is NR¹ or CHR¹;
R¹ is hydrogen, (C₁-C₆)alkyl or (C₁-C₆)alkylphenyl when ring A is saturated (i.e., when ring A contains no double bonds) and R¹ is absent when ring A contains a double bond;
R² is selected from naphtha, phenyl, (C₁-C₆)alkylphenyl, 1-adamantyl, 2-adamantyl, (C₁-C₈) straight or branched alkyl, (C₃-C₁₀)cycloalkyl and (C₈-C₃₀)bicyclic or tricyclic alkyl; wherein said (C₃-C₁₀)cycloalkyl and said (C₈-C₃₀)bicyclic or tricyclic alkyl may optionally be substituted with a hydroxy group; and wherein said adamantyl groups may optionally be substituted with from one to three substituents independently selected from (C₁-C₆)alkyl, halo and hydroxy; and
R³ and R⁴ are independently selected from benzyl, wherein the phenyl moiety of said benzyl may optionally be substituted with an amino or nitro group; hydrogen, phenyl, (N≡C)—(C₁-C₆)alkyl, (C₁-C₆)alkyl-O—C(═O)—(C₁-C₆)alkyl and Het-CH₂, wherein Het is selected from 2-, 3- or 4-pyridinyl, furyl, tetrahydrofuryl, pyrimidyl, pyrazinyl, pyrazolyl, isoxazolyl, thiophenyl and triazolyl;
with the proviso that (a) no more than one of the two dotted lines can represent a double bond in any one compound, (b) when Z is (H, H), X is CH₂, (c) when Z is oxygen or (H, H) and X is CHR¹, R¹ must be hydrogen, (d) when Z is sulfur and X is NR¹, R¹ must be hydrogen, and (e) one of R³ and R⁴ must be Het-CH₂, and
(d) the compound 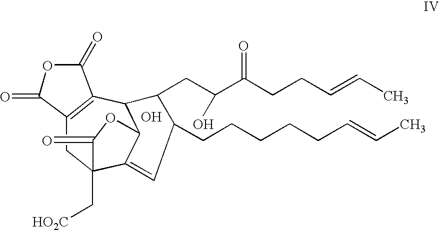
and the pharmaceutically acceptable salts of the foregoing compounds.

Other more specific embodiments of this invention relate to any of the above pharmaceutical compositions and methods of treatment wherein the FTase inhibitor is selected from compounds of the formula I as defined above, wherein R¹ and R² are both (CH₂)ₚ(5-10 membered heterocycles) wherein p is 1 or 2.

Other more specific embodiments of this invention relate to any of the above pharmaceutical compositions and methods of treatment wherein the FTase inhibitor is selected from compounds of the formula I as defined above, wherein R³ is a (CH₂)ₘ(pinane) wherein m is 0, 1 or 2, and, more preferably, those wherein R³ is pinanemethyl.

Other more specific embodiments of this invention relate to any of the above pharmaceutical compositions and methods of treatment wherein the FTase inhibitor is selected from compounds of the formula 1, as defined above, wherein R³ is 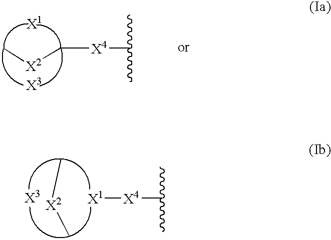
wherein X¹, X², X³ and X⁴ are as defined above.

Other more specific embodiments of this invention relate to any of the above pharmaceutical compositions and methods of treatment wherein the FTase inhibitor is selected from compounds of the formula 1, as described above, wherein R⁴ is phenyl optionally substituted by 1 to 3 R⁵ substituents.

Other more specific embodiments of this invention relate to any of the above pharmaceutical compositions and methods of treatment, wherein the FTase inhibitor is selected from the compounds listed below:
2-[2-(4-Bromo-phenyl)-2-oxo-ethylidene]-5,5-bis-pyridin-4-ylmethyl-3-(2,6,6-trimethyl-bicyclo[3.1.1]hept-3-ylmethyl)-imidazolidin-4-one;
4-{[5-Oxo-4,4-bis-pyridin-4-ylmethyl-1-(2,6,6-trimethyl-bicyclo[3. 1.1]hept-3-ylmethyl)-imidazolidin-2-ylidene]-acetyl}-benzonitrile;
2-[2-(4-Chloro-phenyl)-2-oxo-ethylidene]-5,5-bis-pyridin-4-ylmethyl-3-(2,6,6-trimethyl-bicyclo[3.1.1]hept-3-ylmethyl)-imidazolidin4-one;
2-[2-(3,4-Dichloro-phenyl )-2-oxo-ethylidene]-5,5-bis-pyridin-4-ylmethyl-3-(2,6,6-trimethyl-bicyclo[3.1.1]hept-3-ylmethyl)-imidazolidin4-one;
2-[2-(3-Nitro-phenyl)-2-oxo-ethylidene]-5,5-bis-pyridin-4-ylmethyl-3-(2,6,6-trimethyl-bicyclo[3.1.1]hept-3-ylmethyl)-imidazolidin4-one;
2-[2-(4-Methoxy-phenyl)-2-oxo-ethylidene]-5,5-bis-pyridin4-ylmethyl-3-(2,6,6-trimethyl-bicyclo[3.1.1]hept-3-ylmethyl)-imidazolidin4-one;
2-[2-(3-Methoxy-phenyl)-2-oxo-ethylidene]-5,5-bis-pyridin-4-ylmethyl-3-(2,6,6-trimethyl-bicyclo[3.1.1]hept-3-ylmethyl)-imidazolidin-4-one;
2-[2-(2-Methoxy-phenyl)-2-oxo-ethylidene]-5,5-bis-pyridin4-ylmethyl-3-(2,6,6-trimethyl-bicyclo[3.1.1]hept-3-ylmethyl)-imidazolidin4-one;
2-(2-Biphenyl-4-yl-2-oxo-ethylidene)-5,5-bis-pyridin-4-ylmethyl-3-(2,6,6-trimethyl-bicyclo[3.1.1]hept-3-ylmethyl)-imidazolidin4-one;
2-(2-Naphthalen-2-yl-2-oxo-ethylidene)-5,5-bis-pyridin-4-ylmethyl-3-(2,6,6-trimethyl-bicyclo[3.1.1]hept-3-ylmethyl)-imidazolidin4-one;
2-[2-(4-Fluoro-phenyl)-2-oxo-ethylidene]-5,5-bis-pyridin4-ylmethyl-3-(2,6,6-trimethyl-bicyclo[3.1.1]hept-3-ylmethyl)-imidazolidin4-one;
2-[2-(2,4-Difluoro-phenyl)-2-oxo-ethylidene]-5,5-bis-pyridin-4-ylmethyl-3-(2,6,6-trimethyl-bicyclo[3.1.1]hept-3-ylmethyl)-imidazolidin4-one;
4-{[5-Oxo-4,4-bis-pyridin-4-ylmethyl-1-(2,6,6-trimethyl-bicyclo[3.1.1]hept-3-yl)-imidazolidin-2-ylidene]-acetyl}-benzonitrile;
2-[2-(4-Nitro-phenyl)-2-oxo-ethylidene]-5,5-bis-pyridin-4-ylmethyl-3-(2,6,6-trimethyl-bicyclo[3.1.1]hept-3-ylmethyl)-imidazolidin-4-one;
2-[2-Oxo-2-phenyl-ethylidene]-5,5-bis-pyridin-4-ylmethyl-3-(2,6,6-trimethyl-bicyclo[3.1.1]hept-3-ylmethyl)-imidazolidin-4-one;
2-{2-Oxo-2-[4-(2H-tetrazol-5-yl)-phenyl]-ethylidene}-5,5-bis-pyridin-4-ylmethyl-3-(2,6,6-trimethyl-bicyclo[3.1.1]hept-3-ylmethyl)-imidazolidin-4-one;
3-{[5-Oxo-4,4-bis-pyridin-4-ylmethyl-1-(2,6,6-trimethyl-bicyclo[3.1.1]hept-3-ylmethyl)-imidazolidin-2-ylidene]-acetyl}-benzonitrile;
4-{[5-Oxo-4,4-bis-pyridin-4-ylmethyl-1-(2,6,6-trimethyl-bicyclo[3.1.1]hept-3-ylmethyl)-imidazolidin-2-ylidene]-acetyl}-benzoic acid ethyl ester;
2-[2-Oxo-2-(4-trifluoromethyl-phenyl)-ethylidene]-5,5-bis-pyridin-4-ylmethyl-3-(2,6,6-trimethyl-bicyclo[3.1.1]hept-3-ylmethyl)-imidazolidin4-one;
2-[2-(4-Methanesulphonyl-phenyl)-2-oxo-ethylidene]-5,5-bis-pyridin-4-ylmethyl-3-(2,6,6-trimethyl-bicyclo[3.1.1]hept-3-ylmethyl)-imidazolidin-4-one;
4-{[1 -(6,6-Dimethyl-bicyclo[3.1.1]hept-2-ylmethyl)-5-oxo4,4-bis-pyridin-4-ylmethyl-imidazolidin-2-ylidene]-acetyl}-benzonitrile;
4-[(1-Bicyclo[2.2.2]oct-1-ylmethyl-5-oxo4,4-bis-pyridin-4-ylmethyl-imidazolidin-2-ylidene)-acetyl]-benzonitrile;
4-{[1 -(2-Ethyl-6,6-dimethyl-bicyclo[3.1.1]hept-3-ylmethyl)-5-oxo-4,4-bis-pyridin-4-ylmethyl-imidazolidin-2-ylidene]-acetyl}-benzonitrile;
4-{[1-(2-Benzyl-6,6-dimethyl-bicyclo[3.1.1]hept-3-ylmethyl)-5-oxo-4,4-bis-pyridin-4-ylmethyl-imidazolidin-2-ylidene]-acetyl}-benzonitrile;
4-{[1-(2-Isopropenyl-6,6-dimethyl-bicyclo[3.1.1]hept-3-ylmethyl)-5-oxo-4,4-bis-pyridin-4-ylmethyl-imidazolidin-2-ylidene]-acetyl}-benzonitrile;
4-{[1-(2-isopropyl-6,6-dimethyl-bicyclo[3.1.1]hept-3-ylmethyl)-5-oxo-4,4-bis-pyridin-4-ylmethyl-imidazolidin-2-ylidene]-acetyl}-benzonitrile;
4-({1-[2-(1-Methoxyimino-ethyl)-6,6-dimethyl-bicyclo[3.1.1]hept-3-ylmethyl]-5-oxo-4,4-bis-pyridin-4-ylmethyl-imidazolidin-2-ylidene}-acetyl)-benzonitrile;
4-{[1-(6,6-Dimethyl-2-methylene-bicyclo[3.1.1]hept-3-ylmethyl)-5-oxo4,4-bis-pyridin-4-ylmethyl-imidazolidin-2-ylidene]-acetyl}-benzonitrile;
4-{[1-(2-Hydroxy-2-hydroxymethyl-6,6-dimethyl-bicyclo[3.1.1]hept-3-ylmethyl)-5-oxo-4,4-bis-pyridin-4-ylmethyl-imidazolidin-2-ylidene]-acetyl}-benzonitrile;
4-{[1-(6,6-Dimethyl-2-oxo-bicyclo[3.1.1]hept-3-ylmethyl)-5-oxo4,4-bis-pyridin-4-ylmethyl-imidazolidin-2-ylidene]-acetyl}-benzonitrile;
3-tert-Butyl-2-(2-oxo-2-phenyl-ethylidene)-5,5-bis-pyridin-4-yl methyl-imidazolidin-4-one;
4-{[1-(2,2-Dimethyl-propyl )-5-oxo-4,4-bis-pyridin-4-ylmethyl-imidazolidin-2-ylidene]-acetyl}-benzonitrile;
4-{[1-(2-Adamantan-1-yl-ethyl)-5-oxo4,4-bis-pyridin-4-ylmethyl-imidazolidin-2-ylidene[-acetyl}-benzonitrile;
3-Cyclohexyl-2-(2-oxo-2-phenyl-ethylidene)-5,5-bis-pyridin-4-ylmethyl-imidazolidin-4-one;
4-[(1 -Adamant-1 -ylmethyl-5-oxo-4,4-bis-pyridin-4-ylmethyl-imidazolidin-2-ylidene)-acetyl]-benzonitrile;
4-[(1-Cyclohexylmethyl-5-oxo-4,4-bis-pyridin-4-ylmethyl-imidazolidin-2-ylidene)-acetyl]-benzonitrile;
3-Hexyl-2-(2-oxo-2-phenyl-ethylidene)-5,5-bis-pyridin-4-ylmethyl-imidazolidin-4-one;
3-Napthalen-1-yl-2-(2-oxo-2-phenyl-ethylidene)-5,5-bis-pyridin4-ylmethyl-imidazolidin-4-one;
3-Adamantan-1-yl-2-(2-oxo-2-phenyl-ethylidene)-5,5-bis-pyridin-4-ylmethyl-imidazolidin-4-one;
3-Adamantan-1-yl-2-[2-(4-nitro-phenyl)-2-oxo-ethylidene]-5,5-bis-pyridin-4-ylmethyl-imidazolidin-4-one;
4-[(1-Benzyl-5-oxo-4,4-bis-pyridin-4-ylmethyl-imidazolidin-2-ylidene)-acetyl]-benzonitrile;
4-[(1-Allyl-5-oxo-4,4-bis-pyridin-4-ylmethyl-imidazolidin-2-ylidene)-acetyl]-benzonitrile;
4-[( 1-Methyl-5-oxo-4,4-bis-pyridin4-ylmethyl-imidazolidin-2-ylidene)-acetyl]-benzonitrile;
4-{[1-(2,2-Diethoxy-ethyl)-5-oxo-4,4-bis-pyridin-4-ylmethyl-imidazolidin-2-ylidene]-acetyl}-benzonitrile; 4-[(1-Adamantan-2-ylmethyl-5-oxo-4,4-bis-pyridin-4-ylmethyl-imidazolidin-2-ylidene)-acetyl]-benzonitrile;
4-[( 1-Adamantan-2-yl-5-oxo-4,4-bis-pyridin-4-ylmethyl-imidazolidin-2-ylidene)-acetyl]-benzonitrile;
4-[(5-Oxo-1-phenyl-4,4-bis-pyridin-4-ylmethyl-imidazolidin-2-ylidene)-acetyl]-benzonitrile; and,
4-{[4-tert-Butyl-phenyl-5-oxo4,4-bis-pyridin-4-ylmethyl-imidazolidin-2-ylidene)-acetyl]-benzonitrile.
and the pharmaceutically acceptable salts of such compounds.

Other more specific embodiments of this invention relate to any of the above pharmaceutical compositions and methods of treatment wherein the HMG CoA reductase inhibitor contained in such composition or used in such method is selected from the group consisting of atorvastatin, pravastatin, niacin, gemfibrozil, clofibrate, lovastatin, fluvastatin, simvastatin and compactin, and the pharmaceutically acceptable salts of the foregoing compounds.

Other more specific embodiments of this invention relate to any of the above pharmaceutical compositions and methods of treatment wherein the HMG CoA reductase inhibitor contained in such composition or used in such method is atorvastatin.

Other more specific embodiments of this invention relate to any of the above pharmaceutical compositions and methods of treatment wherein the HMG CoA reductase inhibitor contained in such composition or used in such method is lovastatin.

Other more specific embodiments of this invention relate to any of the above the pharmaceutical compositions and methods of treatment wherein the FTase inhibitor contained in such composition or used in such method is selected from:
(a) compounds of the formula IIA, as defined above, wherein R³ is 4-pyridyl, 4-pyrimidyl or 2-fluoro-4-pyridyl;
(b) compounds of the formula IIA, as defined above, wherein R² is —(CH₂)ₙY;
(c) compounds of the formula IIA, as defined above, wherein R² is —(CH₂)ₙY and n is an integer from 1 to 5;
(d) compounds of the formula IIA, or IIB as defined above, wherein each of R¹, E¹, E² and R⁴, if present, is hydrogen; and
(e) compounds of the formula IIA, as defined above, wherein R² is —(CH₂)ₙ—Y, R¹is 4-pyridyl, 4-pyrimidyl or 2-fluoro-4-pyridyl, R⁵ is (C₁-C₂) alkyl and Y is —CO₂R⁵, cyano, —CONHR⁴, CH═CHCO₂R⁵ or —OCOR⁵;

Other more specific embodiments embodiments of this invention relate to any of the above pharmaceutical compositions and methods of treatment wherein the FTase inhibitor contained in such composition or used in such method is not limonene or d-limonene.

The term “alkyl”, as used herein, unless otherwise indicated, includes saturated monovalent hydrocarbon radicals having straight, branched or cyclic moieties or combinations thereof.

The term “halo”, as used herein, refers to chloro, fluoro, bromo or iodo.

The above compounds of the formulas I, IIA, IIB, III and IV may contain one or more chiral centers and therefore may exist in 2 or more enantiomeric and diastereomeric forms. The above definitions of the compounds having formulas I, IA, IIB, III and IV include all enantiomers, diasteriomers and other stereoisomers of these compounds, as well as mixtures thereof.

The following references refer to compounds that exhibit activity as FTase inhibitors and which can be used, in combination with an HMG CoA reductase inhibitor, in the pharmaceutical compositions and methods of this invention, and to methods of preparing the same: International Patent Application PCT/US92/11292, which designates the United States and was published on Jul. 22, 1993 as WO 93/14085; U.S. Pat. No. 4,876,259, which issued on Oct. 24, 1989; U.S. Pat. No. H1345 which issued on Aug. 2, 1994; U.S. Pat. No. 5,260,332, which issued on Nov. 9, 1993; U.S. Pat. No. 5,262,435, which issued on Nov. 16, 1993; U.S. Pat. No. 5,369,125, issued on Nov. 29, 1994; World Patent Application WO 93/24633, which was published on Dec. 9, 1993: World Patent Application WO 94/03597, which was published on Feb. 17, 1994; World Patent Application WO 94/16069, which was published on Jun. 21, 1994; G. L. Bulton, et al., 209th American Chem. Soc. Nat'l Meeting, Anaheim, Calif., Apr. 2-6, 1995, Division of Med. Chem., Abs. No. 032, World Patent Application WO 95/00497, which was published on Jan. 5, 1995; U.S. Pat. No. 5,260,479, which was published on Nov. 9, 1993; World Patent Application WO 95/10514; World Patent Application WO 95/10515; World Patent Application WO 95/10516; World Patent Application WO 95/12572, which was published on May 11, 1995; World Patent Application WO 95/11917, which was published on May 4, 1995; World Patent Application WO 94/26723, which was published on Nov. 24, 1994; World Patent Application WO 95/25086, which was published on Sep. 21, 1995; Kanda et al, AFMC. International Medicinal Chemistry Symposium AIMECS 95, Tokyo, Japan, Poster, P7M153, Sept. 4, 1995; World Patent Application WO 96/10037 which was published on Apr. 4, 1996; World patent Application 96/10035, which was published on Apr. 4, 1996; World Patent Application WO 96/10034, which was published on Apr. 4,1996; World Patent Application WO 96/10011, which was published on Apr. 6, 2996; World Patent Application WO 96/10011, which was published on Apr. 6, 1996; World Patent Application WO 96/09821, which was published on Apr. 4, 1996; World Patent Application WO 96/09820, which was published on Apr. 4, 1996; Quin et al, 211th American Chemical Society National Meeting, New Orleans, La., Mar. 24-28, 1996, Lecture, COMP 012, Mar. 24, 1996; World Patent Applications WO 96/06609 and WO 96/06604, both of which were published on Mar. 7, 1996; European Patent Application EP 696,593, which was published on Feb. 14, 1996; Hartman, G. D., 14th International Symposium on Medicinal Chemistry, Maastricht, Netherlands, Sep. 8-12, 1996, Lectura, SL-08.3, Sep. 10, 1996; World Patent Application WO 96/30363, which was published on Oct. 3, 1996; World Patent Application WO 96/30343, which was published in Oct. 3, 1996, World Patent Application WO 97/03050; World Patent Application WO 94/26723, which was published on Nov. 24, 1994; International Patent Application PCT/IB95/00189, which designates the United States and was filed on Mar. 20 1995; U.S. patent application Ser. No. 08/236,743, which was filed on Apr. 29, 1994; U.S. Provisional Application entitled “Adamantyl Substituted Oxindoles As Pharmaceutical Agents,” which was filed on May 28, 1996, in the name of R. A. Volkmann and J. P. Lyssikatos; U.S. Pat. No. 5,350,867, which issued on Sep. 27, 1994; U.S. Pat. No. 5,352,705, which issued on Oct. 4, 1994; U.S. Pat. No. 5,565,489, which issued on Oct. 15, 1996; European Patent Application EP 750,609, which was published on Jan. 2, 1997; European Patent Application 461,869, which was published on Dec. 18, 1991; and World Patent Application 96/21456, which was published on Jul. 18, 1996.

The following references refer to compounds that exhibit activity as HMG CoA reductase inhibitors and which can be used, in combination with a FTase inhibitor, in the pharmaceutical compositions and methods of this invention, and to methods of preparing the same: U.S. Pat. No. 4,681,893, issued Jul. 21, 1987; U.S. Pat. No. 5,273,995, issued Dec. 28, 1993; U.S. Pat. No. 5,385,929, issued Jan. 31, 1995; U.S. Pat. No. 4,957,971, issued Sep. 18, 1990; U.S. Pat. No. 5,102,893, issued Apr. 7, 1992; U.S. Pat. No. 4,957,940, issued Sep. 18, 1990; U.S. Pat. No. 4,950,675, issued Aug. 21, 1990; U.S. Pat. No. 4,929,620, issued May 29, 1990; U.S. Pat. No. 4,923,861, issued May 8, 1990; U.S. Pat. No. 4,906,657, issued Mar. 6, 1990; U.S. Pat. No. 4,868,185, issued Sep. 19, 1989; U.S. Pat. No. 5,124,482 issued Jun. 23, 1992; U.S. Pat. No. 5,003,080, issued Mar. 26, 1991; U.S. Pat. No. 5,097,045, issued Mar. 17, 1992; U.S. Pat. No. 5,149,837, issued Sep. 22, 1992; U.S. Pat. No. 4,906,624, issued Mar. 6, 1990; U.S. Pat. No. 4,761,419, issued Aug. 2, 1988; U.S. Pat. No. 4,735,950, issued Apr. 5, 1988; U.S. Pat. No. 4,808,621, issued Feb. 28, 1989; U.S. Pat. No. 4,647,576, issued Mar. 3, 1987; U.S. Pat. No. 5,118,882, issued Jun. 2, 1992; U.S. Pat. No. 5,214,197, issued May 25, 1993; U.S. Pat. No. 5,321,046, issued Jun. 14, 1994; U.S. Pat. No. 5,260,440, issued Nov. 9, 1993; and U.S. Pat. No. 5,208,258 issued May 4, 1993; U.S. Pat. No. 5,369,125, issued Nov. 29, 1994; U.S. Pat. No. H1345 issued Aug. 2, 1994; U.S. Pat. No. 5,262,435, issued Nov. 16, 1993; and U.S. Pat. No. 5,260,332, issued Nov. 9, 1993. Great Britian Patent Application GB 2,055,100, published Feb. 25, 1981; U.S. Pat. No. 4,499,289, issued Feb. 12, 1983; U.S. Pat. No. 4,645,854, issued Feb. 24, 1987; U.S. Pat. No. 4,613,610 issued Sep. 23, 1986; U.S. Pat. No. 4,668,699, issued May 26, 1987; U.S. Pat. No. 4,851,436, issued Jul. 25, 1989; U.S. Pat. No. 4,678,806, issued Jul. 7, 1987; U.S. Pat. No. 4,772,626, issued Sep. 20, 1988; U.S. Pat. No. 4,855,321, issued Aug. 8, 1989; European Patent Application EP 244364, published Nov. 4, 1987; U.S. Pat. No. 4,766,145, issued Aug. 23, 1988; U.S. Pat. No. 4,876,279, issued Oct. 24, 1989; U.S. Pat. No. 4,847,306, issued Jul. 11, 1989; U.S. Pat. No. 5,049,696, issued Sep. 17, 1991; European Patent Application EP 245,990, published Nov. 19, 1987; European Patent Application EP 251,625, published Jan. 7, 1988; U.S. Pat. No. 4,719,229, published Jan. 12, 1988; Japanese Patent Application 63014722, published Jan. 21, 1988; U.S. Pat. No. 4,736,064, issued Apr. 5, 1988; U.S. Pat. No. 4,738,982 issued Apr. 19, 1988; U.S. Pat. No. 4,845,237, issued Jul. 4, 1989; European Patent EP 306,263, granted Mar. 18, 1992; U.S. Pat. No. 5,026,708, issued Jun. 25, 1991; U.S. Pat. No. 4,863, 957, issued Sep. 5, 1989; U.S. Pat. No. 4,946,841, issued Aug. 7, 1990; European Patent 339358, granted Jul. 13, 1994; U.S. Pat. No. 4,937,264 issued Jun. 26, 1998; U.S. Pat. No. 4,876,366, issued Oct. 24, 1989; U.S. Pat. No. 4,921,974, issued May 1, 1990; U.S. Pat. No. 4,963,538 issued Oct. 16, 1990; U.S. Pat. No. 5,130,306, issued Jul. 14, 1992; U.S. Pat. No. 4,900,754 issued Feb. 13, 1990; U.S. Pat. No. 5,026,698, issued Jun. 25, 1991; U.S. Pat. No. 4,977,161, issued Dec. 11, 1990; U.S. Pat. No. 4,927,851, issued May 22, 1990; European Patent Application EP 373,507, published Jun. 20, 1990; U.S. Pat. No. 4,939,143, issued Jul. 3, 1990; U.S. Pat. No. 4,939,159, issued Jul. 3, 1990; U.S. Pat. No. 4,940,727, issued Jul. 10, 1990; U.S. Pat. No. 5,116,870, issued May 26, 1992; Australian Patent AU 635,545, granted Mar. 25, 1993; U.S. Pat. No. 5,098,391, issued Mar. 24, 1992; U.S. Pat. No. 5,294,724, issued Mar. 15, 1994; U.S. Pat. No. 5,001,255, issued Mar. 19, 1991; U.S. Pat. No. 5,149,834, issued Sep. 22, 1992; U.S. Pat. No. 5,089,523, issued Feb. 18, 1992; European Patent Application EP 465,265 published Jan. 8, 1992; U.S. Pat. No. 5,476,846, issued Dec. 19, 1995; U.S. Pat. No. 5,321,046, issued Jun. 14, 1994; U.S. Pat. No. 5,106,992, issued Apr. 21, 1992; U.S. Pat. No. 5,347,039, issued Sep. 13, 1994; Japanese Patent Application 4193836, published Jul. 13, 1992; Great Britian patent Application 2253787, published Sep. 23, 1992; U.S. Pat. No. 5,411,969, issued May 2, 1995; Japanese Patent Application 4,356,435, published Dec. 10, 1992; U.S. Pat. No. 5,266,707 issued Nov. 30, 1993; U.S. Pat. No. 5,455,247 issued Oct. 3, 1995; U.S. Pat. No. 5,475,029, issued Dec. 12, 1995; U.S. Pat. No. 5,591,772, issued Jan. 7, 1997; U.S. Pat. No. 5,286,746 issued Feb. 15, 1994; Japanese Patent Application JP 7089898, published Apr. 4, 1995; European Patent Application EP 677,039, published Oct. 18, 1995 and World Patent Application 96/08248, published Mar. 21, 1996.

This invention relates both to methods of treating cancer in which the FTase inhibitor and the HMG CoA reductase inhibitor are administered together, as part of the same pharmaceutical composition, as well as to methods in which these two active agents are administered separately as part of an appropriate dose regimen designed to obtain the benefits of the combination therapy. The appropriate dose regimen, the amount of each dose administered, and specific intervals between doses of each active agent will depend upon the subject being treated, the type of cancer or abnormal cell growth and the severity of the condition. In carrying out the methods of this invention, the FTase inhibitor will be administered in the amounts disclosed in the literature, or otherwise believed to be effective, for the administration of such compound as a single active agent for the treatment of cancer or the inhibition of abnormal cell growth, and the HMG CoA reductase inhibitor will be administered in an amount that is about one quarter to one half of the amount disclosed in the literature, or otherwise believed to be effecive, for administration of such compound as a single agent for the treatment of hypercholesterolemia. For example, in carrying out the present inventions, the FTase inhibitors of formulas I, IIA, IIB and III will typically be admisterered to an average 70 kg adult human in an amount ranging from about 0.005 to about 0.6 mg per kg body weight of the subject being treated per day, in single or divided doses, and the HMG CoA reductase inhibitor atorvastatin will typically be administered in an amount ranging from about 0.07 to about 3.6 mg per kg body weight per day, in single or divided doses. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the above range may be more than adequate, while in other cases dosage levels higher than the above upper daily limit may be employed without causing any harmful side effect, provided that such larger dosages are administered as several small doses for administration throughout the day.

The FTase inhibitors and the HMG CoA reductase inhibitors that are employed in the pharmaceutical compositions and methods of this invention are hereinafter also referred to as “therapeutic agents”. The therapeutic agents can be administered via either the oral or parenteral route. Compositions containing both a FTase inhibitor and an HMG CoA reductase inhibitor will generally be administered orally or parenterally daily, in single or divided doses, so that the total amount of each active agent administered falls within the above guidelines.

The therapeutic agents may be administered alone or in combination with pharmaceutically acceptable carriers or diluents by either of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutic compounds of this invention, when administered separately (i.e., not in the same pharmaceutical composition) are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a therapeutic agent in either sesame or peanut oil or in aqueous propylene glycol may be employed. The aqueous solutions should be suitably buffered if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

The activity of the therapeutic compounds as FTase inhibitors may be determined by their ability, relative to a control, to inhibit Ftase in vitro. This procedure is described below.

A crude preparation of FTase comprising the cytosolic fraction of homogenized brain tissue is used for screening compounds in a 96-well assay format. The cytosolic fraction is prepared by homogenizing approx. 40 grams fresh tissue in 100 ml of sucrose/MgCl₂/EDTA buffer (using a Dounce homogenizer; 10-15 strokes), centrifuging the homogenates at 1000 grams for 10 minutes at 4G, re-centrifuging the supernatant at 17,000 grams for 15 minutes at 4G, and then collecting the resulting supernatant. This supernatant is diluted to contain a final concentration of 50 mM Tris HCl (pH 7.5), 5 mN DTT, 0.2 M KCl, 20 mM ZnCl₂, 1 mM PMSF and re-centrifuged at 178,000 grams for 90 minutes at 4G. The supernatant, termed “crude FTase” was assayed for protein concentration, aliquoted, and stored at −70° C.

The assay used to measure in vitro inhibition of human FTase is a modification of the method described by Amersham LifeScience for using their Farnesyl transferase (3H) Scintilation Proximity Assay (SPA) kit (TRKQ 7010). FTase enzyme activity is determined in a volume of 100 ml containing 50 mM N-(2-hydroxy ethyl) piperazine-N¢-(2-ethane sulfonic acid) (HEPES), pH 7.5, 30 mM MgCl₂, 20 uM KCl, 5 mM Na₂HPO₄, 5 mM dithiothreitol (DTT), 0.01% Triton X-100, 5% dimethyl sulfoxide (DMSO), 20 mg of crude FTase, 0.12 mM [3H]-farnesyl pyrophosphate ([3H]-FPP; 36000 dpm/pmole, Amersham LifeScience), and 0.2 mM of biotinylated Ras peptide KTKCVIS (Bt-KTKCVIS) that is N-terminally biotinylated at its alpha amino group and was synthesized and purified by HPLC in house. The reaction is initiated by addition of the enzyme and terminated by addition of EDTA (supplied as the STOP reagent in kit TRKQ 7010) following a 45 minute incubation at 37° C. Prenylated and unprenylated Bt-KTKCVIS is captured by adding 10 ml of steptavidin-coated SPA beads (TRKQ 7010) per well and incubating the reaction mixture for 30 minutes at room temperature. The amount of radioactivity bound to the SPA beads is determined using a MicroBeta 1450 plate counter. Under these assay conditions, the enzyme activity is linear with respect to the concentrations of the prenyl group acceptor, Bt-KTKCVIS, and crude FTase, but saturating with respect to the prenyl donor, FPP. The assay reaction time is also in the linear range.

The test compounds are routinely dissolved in 100% DMSO. Inhibition of farnesyl transferase activity is determined by calculating percent incorporation of tritiated-farnesyl in the presence of the test compound vs. its incorporation in control wells (absence of inhibitor). IC₅₀ values, that is, the concentration required to produce half maximal farnesylation of Bt-KTKCVIS, is determined from the dose-responses obtained.

A fluorescence assay for FTase activity that can be used to screen for FTase inhibitors is described in UK Patent Application GB 2,267,966, which was published on Dec. 22, 1993.

The activity of certain therapeutic agents as HMG CoA reductase inhibitors may be determined by the procedure described by Dugan et al, Achiv. Biochem. Biophys., (1972), 152, 21-27. In this method, the level of HMG-CoA enzyme activity in standard laboratory rats is increased by feeding the rats a chow diet contining 5% cholestyramine for four days, after which the rats are sacrificed. The rat livers are homogenized, and the incorporation of cholesterol- ⁴C-acetate into nonsaponifiable lipid by the rat liver homogenate is measured. The micromolar concentration of compound required for 50% inhibition of sterol synthesis over a one-hour period is measured, and expressed as an IC₅₀ value.

A second method (designated COR screen) is that described by T. Kita, et al, J. Clin. Invest., (1980), 66: 1094-1100. In this method, the amount of ¹⁴C-HMG-CoA converted to ¹⁴C-mevalonate in the presence of a purified enzyme preparation of HMG-CoA reductase is measured. The micromolar concentration of compound required for 50% inhibition of cholesterol synthesis is measured and recorded as an IC₅₀ value.

The various methods of this invention may be practiced as part of a therapy that includes the administration of one or more other anti-tumor substances, for example, those selected from mitotic inhibitors, for example, vinblastine; alkylating agents, for example, cisplatin, carboplatin and cyclophosphamide; antimetabolites, for example, 5-fluorouracil, cystosine arabinoside and hydroxyurea, or, for example, one of the preferred antimetabolites disclosed in European Patent Application No. 239362 such as N-{5-[N-(3,4-dihydro-2-methyl-4-oxoquinazolin-6-ylmethyl)-N-methylamino]-2-thenoyl}-L-glutamic acid; intercalating antibiotics, for example, adriamycin and bleomycin; enzymes, for example, asparaginase; topoisomerase inhibitors, for example, etoposide; biological response modifiers, for example, interferon; and anti-hormones, for example, antioestrogens such as ‘NOLVADEX’ (tamoxifen) or antiandrogens such as ‘CASODEX’ (4′-cyano-3-(4-fluorophenylsulphonyl)-2-hydroxy-2-methyl-3′-(trifluoromethyl)propionanilide. Such therapies may be achieved by way of the simultaneous, sequential or separate dosing of the individual components of the therapy. According to this aspect of the invention, there is provided a pharmaceutical product comprising a pharmaceutically acceptable carrier, as described above, one or both of an HMG CoA reductase inhibitor and a FTase inhibitor, and an additional anti-tumor agent, as described above.

As indicated in Table 1 below, the present inventor has shown that the effectiveness of Compound 1, which has the structure

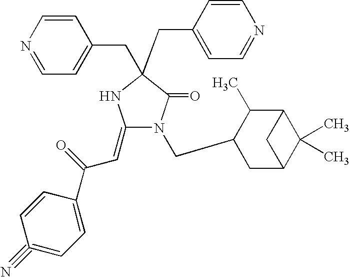

can be enhanced by a minimally effective dose of lovastatin.

TABLE 1
Synergistic Effects of Lovastatin and Compound 1 Treatment on
Prenylation of K-ras 4B in Intact Cells

% Inhibition OF K-Ras 4B
Prenylation*

| Compound 1 [μm] | CONTROL | +5 μM Lovastatin |
|---|---|---|
| 0 | 0 | 23 |
| 0.1 | 0 | 56 |
| 1.0 | 0 | 83 |
| 10 | 0 | 96 |
| *Semi-confluent monolayers of the NIH-3T3 tranfectant overexpressing mutant K-Ras 4B were treated for 18 hours at 37° C. with increasing concentrations of CP-390,392 in the presence and absence of 5 μM of hydrolysed lovastatin. Cells were lysed in a RIPA lysis buffer (50 mM tris[hydroxymethyl] amino-methane, 0.15 M sodium chloride, 1% sodium deoxycholate, 1% Triton X-100, 0.1% SDS, 0.25 sodium azide; ph 8.5) containing 1 mM of DTT (dithiothreitol; Boehringer | | |
| #Mannheim, Indianapolis, IN) and protease inhibitors (Aprotinin, Leupeptin, Anitpain, Pefabloc at final concentrations of 10 μg/ml, 2 μg/ml, 2 μg/ml and 50 μM, respectively; Boehringer Mannheim, Indianapolis, IN) and boiled for 3 minutes. Equal amounts of protein (100 μg/lane) were resolved by SDS-PAGE on 12.5% gels and transferred to Immobilon-P membranes (Intergrated Separation Systems, Natick, MA.). The membranes were immunoblotted with 5 μg/ml of | | |
| #anti-Pan-ras (Ab-3) monoclonal antibody (Calbiochem, La Jolla, CA). The blots were incubated with peroxidase-conjugated secondary antibody, and the immunoblotted Ras protein were detected by enhanced chemiluminescence (Amersham Life Products, Arlington Heights, IL). Percent of prenylated Ras was determined by densitometric scanning using MasterScan 3.0 (Scanalytics, Billerica, Massachusettes). | | |

1. A pharmaceutical composition for the treatment of cancer or a benign proliferative disorder in a mammal, comprising a FTase inhibitor, an HMG CoA reductase inhibitor and a pharmaceutically acceptable carrier, wherein the FTase inhibitor and the HMG CoA reductase inhibitor are present in amounts that render the composition effective in the treatment of cancer or a benign proliferative disorder

2. A pharmaceutical composition according to claim 1, wherein the FTase inhibitor is selected from: (a) compounds of the formula 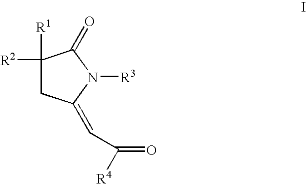
wherein R¹ and R² are independently selected from the group consisting of —(CH₂)ₚ(5-10 membered heterocycles), —(CH₂)ₚ(C₆-C₁₀ aryl), allyl, propargyl and C₁-C₆ alkyl wherein p is 0 to 3, said alkyl and the alkyl moieties of said R¹ and R² groups are optionally substituted by 1 to 3 R⁹ substituents, and the aryl and heterocyclic moieties of said R¹ and R² groups are optionally substituted by 1 to 3 substituents independently selected from halo and R⁹;
R³ is —(CH₂)ₘ(1- or 2-adamantyl), —(CH₂)ₘ(C₃-C₁₀ cycloalkyl), —(CH₂)ₘ(C₆-C₁₀ alkyl, 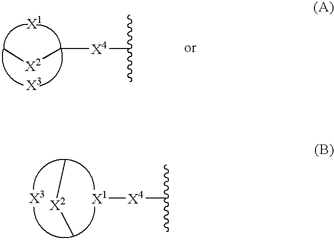
wherein m is 0 to 6, and said cycloalkyl and alkyl optionally contain 1 or 2 double or triple bonds;
X¹, X², and X³ are each independently C₁-C₇ alkylene optionally containing 1 or 2 double or triple bonds, X⁴ is a bond or C₁-C₇ alkylene optionally containing 1 or 2 double or triple bonds, and, in formula (B), the X⁴ moiety is attached to the X¹moiety at any available carbon in the X¹ moiety;
R⁴ is C₆-C₁₀ aryl, 5-10 membered heterocyclyl or C₁-C₆ alkyl wherein each of said R⁴ groups is optionally substituted by 1 to 3 R⁵ substituents;
each R⁵ is independently selected from the group consisting of halo, nitro, cyano, phenyl, —C(O)OR⁶, —SO₂NR⁶R⁷, —NR⁶R⁸, —C(O)R⁶, —OR⁶, —C(O)NR⁶R⁸, —OC(O)NR⁶R⁸, —NR⁸C(O)NR⁸R⁶, —NR⁸C(O)R⁶, —NR⁸C(O)O(C₁-C₄ alkyl), —C(NR⁸)NR⁸R⁶, —C(NCN)NR⁸R⁶, —C(NCN)S(C₁-C₄ alkyl), —NR⁸C(NCN)S(C₁-C₄ alkyl), —NR⁸C(NCN)NR⁸R⁶, —NR⁸SO₂(C₁-C₄ alkyl), —S(O)ₙ(C₁-C₄ alkyl) wherein n is 0 to 2, —NR⁸C(O)C(O)NR⁸R⁶, NR⁸C(O)C(O)R⁸, thiazolyl, imidazolyl, oxazolyl, pyrazolyl, triazolyl tetrazolyl, and C₁-C₄ alkyl optionally substituted by 1 to 3 fluoro substituents;
each R⁶ and R⁷ is independently hydrogen or C₁-C₄ alkyl;
each R³ is independently R⁶ or —OR⁶; and,
each R⁹ is independently selected from cyano, R⁶, —OR⁶, —OC(O)R⁶, —C(O)OR⁶, —C(O)NR⁶R⁷, —NR⁶R⁷, —NR⁶R⁸, —SO₂NR⁶R⁷, and C₁-C₄ alkyl substituted by hydroxy; and
(b) compounds of the formula 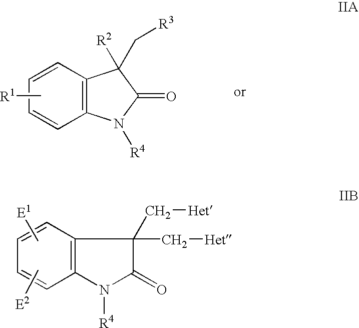
wherein R¹ is hydrogen, halo (e.g., chloro, fluoro, bromo or iodo), cyano, hydroxy, nitro, trifluoromethyl, —NHR⁵, —NR⁵R⁵, R⁵, —OR⁵ or —S(O)ₘ—R⁵;
R² is —(CH₂)ₙ—Y or —OCOR⁵;
R³ is 4-, 3-, or 2-pyridyl, pyrimidyl, pyrazinyl, 2-fluoro4-pyridyl or 3-fluoro-4-pyridyl;
R⁴ is 1-adamantyl or 2-adamantyl;
Y is hydrogen, hydroxy, amino, cyano, —NHR⁵, —NR⁵R⁵, —NHCOR⁵, —NHCO₂R⁵, halo, OR⁵, —S(O)ₘR⁵, —CO₂H, —CO₂R⁵, —CONR⁵R⁵, —CONHR⁵, —CONH₂, —COR⁵, —CH═CHCO₂R⁵, —OCOR⁵, phenyl, phenyl substituted with W, —C≡CCO₂R⁵, —CH═CHR⁵ or —C≡CR⁵;
each R⁵ is, independently, (C₁-C₄) straight or branched alkyl, phenyl or benzyl, wherein said phenyl and the phenyl moiety of said benzyl may optionally be substituted with halo, hydroxy, nitro, cyano, amino, (C₁-C₄) straight or branched alkyl, (C₁-C₄) straight or branched alkoxy, phenyl, benzyl, (C₁-C₄)alkylamino, di[(C₁-C₄)alkyl]amino, or —S(O)ₘ—(C₁-C₄) straight or branched alkyl;
each W is, independently, halo, R⁵, hydroxy, —OR⁵, nitro, amino, —NHR⁵, —NR⁵R⁵, cyano, or —S(O)ₘ—R⁵;
m is 0, 1 or 2;
n is 1 to 7;
p is 0 or 1;
E¹ and E² are selected, independently, from hydrogen, halo, (C₁-C₃)alkyl, hydroxy, (C₁-C₃)alkoxy, nitro, trifluoromethyl, cyano, amino, (C₁-C₃)alkylamino and di[(C₁-C₃)alkyl)amino;
and their pharmaceutically acceptable salts.
Het′ and Het″ are selected, independently, from 6 membered heterocyclic rings containing from one to four nitrogen atoms as part of the ring, optionally substituted with one substituent selected from (C₁-C₃)alkyl, halo, hydroxy, (C₁-C₃)alkoxy, amino, (C₁-C₃)alkylamino and di[(C₁-C₃)alkyl]amino; and
(c) compounds of the formula 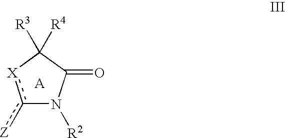
wherein both dotted lines represent optional double bonds;
Z is oxygen or sulfur when it is double bonded to ring A and Z is hydroxy, (C₁-C₁₀)alkyl-S—, (C₁-C₁₀)alkyl-SO—, (C₁-C₁₀)alkyl-SO₂—, adamant-2-yl-S—, naphthyl-S—, benzyl-S—, phenyl-C(═O)CH₂—S—, (C₁-C₆)alkyl-O—C(═O)—CH₂—S— or (H,H) (i.e., Z represents two hydrogen atoms, each of which is single bonded to the same carbon of ring A) when Z is single bonded to ring A, and wherein said naphthyl and phenyl and the phenyl moiety of said benzyl may optionally be substituted with from one to three substituents independently selected from (C₁-C₆)alkyl optionally substituted with from one to three fluorine atoms, (C₁-C₆)alkoxy optionally substituted with from one to three fluorine atoms, halo (e.g., chloro, fluoro, bromo or iodo), amino, (C₁-C₆)alkylamino, [di-(C₁-C₆)alkyl]amino, cyano, nitro, (C₁-C₆)alkyl-SOₙ— wherein n is zero, one or two, —COOH, —COO(C₁-C₆)alkyl and —C(O)NH(C₁-C₆)alkyl;
X is NR¹ or CHR¹;
R¹ is hydrogen, (C₁-C₆)alkyl or (C₁-C₆)alkylphenyl when ring A is saturated (i.e., when ring A contains no double bonds) and R¹ is absent when ring A contains a double bond;
R² is selected from naphthyl, phenyl, (C₁-C₆)alkylphenyl, 1-adamantyl, 2-adamantyl, (C₁-C₈) straight or branched alkyl, (C₃-C₁₀) cycloalkyl and (C₈-C₃₀)bicyclic or tricyclic alkyl; wherein said (C₃-C₁₀)cycloalkyl and said (C₈-C₃₀)bicyclic or tricyclic alkyl may optionally be substituted with a hydroxy group; and wherein said adamantyl groups may optionally be substituted with from one to three substituents independently selected from (C₁-C₆)alkyl, halo and hydroxy; and
R³ and R⁴ are independently selected from benzyl, wherein the phenyl moiety of said benzyl may optionally be substituted with an amino or nitro group; hydrogen, phenyl, (N≡C)—(C₁-C₆)alkyl, (C₁-C₆)alkyl-O—C(═O)—(C₁-C₆)alkyl and Het-CH₂, wherein Het is selected from 2-, 3- or 4-pyridinyl, furyl, tetrahydrofuryl, pyrimidyl, pyrazinyl, pyrazolyl, isoxazolyl, thiophenyl and triazolyl;
with the proviso that (a) no more than one of the two dotted lines can represent a double bond in any one compound, (b) when Z is (H, H), X is CH₂, (c) when Z is oxygen or (H, H) and X is CHR¹, R¹ must be hydrogen, (d) when Z is sulfur and X is NR¹, R¹ must be hydrogen, and (e) one of R³ and R⁴ must be Het-CH₂; and
(d) the compound 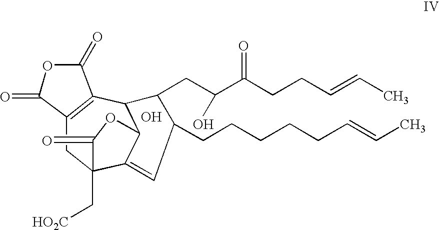
and the pharmaceutically acceptable salts of the foregoing compounds.

3. A pharmaceutical composition according to claim 1, wherein the HMG CoA reductase inhibitor is selected from the group consisting of atorvastatin, pravastatin, lovastatin, compactin fluvastatin and simvastatin, and the pharmaceutically acceptable salts of the foregoing compounds.

4. A method of treating cancer or a benign proliferative disorder in a mammal, comprising administering to said mammal a pharmaceutical composition according to any one of claims 1 to 3.

5. A method of treating cancer or a benign proliferative disorder in a mammal, comprising administering to said mammal a FTase inhibitor and an HMG CoA reductase inhibitor, wherein the FTase inhibitor and the HMG CoA reductase inhibitor are administered in amounts that render the combination of these two active agents effective in treating cancer or a benign proliferative disorder.

6. A method according to claim 5, wherein the HMG CoA reductase inhibitor is atorvastatin, pravastatin, fluvastatin, simvastatin, lovastatin or compactin, or a pharmaceutically acceptable salt thereof.

7. A composition comprising a Farnesyl transferase inhibitor and a hydroxymethylglutaryl coenzyme A reductase inhibitor, wherein said Farnesyl transferase inhibitor is selected from: (a) compounds of the formula 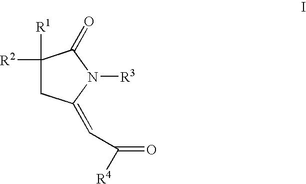
wherein R¹ and R² are independently selected from the group consisting of —(CH₂)ₚ(5-10 membered heterocycles), —(CH₂)ₚ(C₆-C₁ aryl), allyl, propargyl and C₁-C₆ alkyl wherein p is 0 to 3, said alkyl and the alkyl moieties of said R¹ and R² groups are optionally substituted by 1 to 3 R⁹ substituents, and the aryl and heterocyclic moieties of said R¹ and R² groups are optionally substituted by 1 to 3 substituents independently selected from halo and R⁹;
R³ is —(CH₂)ₘ(1- or 2-adamantyl), —(CH₂)ₘ(C₃-C₁₀ cycloalkyl), —(CH₂)ₘ(C₆-C₁₀ alkyl, 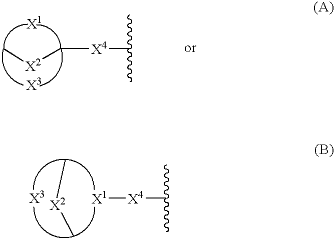
wherein m is 0 to 6, and said cycloalkyl and alkyl optionally contain 1 or 2 double or triple bonds;
X¹, X², and X³ are each independently C₁-C₇ alkylene optionally containing 1 or 2 double or triple bonds, X⁴ is a bond or C₁-C₇ alkylene optionally containing 1 or 2 double or triple bonds, and, in formula (B), the X⁴ moiety is attached to the X¹ moiety at any available carbon in the X¹ moiety;
R⁴ is C₆-C₁₀ aryl, 5-10 membered heterocyclyl or C₁-C₆ alkyl wherein each of said R⁴ groups is optionally substituted by 1 to 3 R⁵ substituents;
each R⁵ is independently selected from the group consisting of halo, nitro, cyano, phenyl, —C(O)OR⁶, —SO₂NR⁶R⁷, —NR⁶R⁸, —C(O)R⁶, —OR⁶, —C(O)NR⁶R⁸, —OC(O)NR⁶R⁸, —NR⁸C(O)NR⁸R⁶, —NR⁸C(O)R⁶, —NR⁸C(O)O(C₁-C₄ alkyl), —C(NR⁸)NR⁸R⁶, —C(NCN)NR⁸R⁶, —C(NCN)S(C₁-C₄ alkyl), —NR⁸C(NCN)S(C₁-C₄ alkyl), —NR⁸C(NCN)NR⁸R⁶, —NR⁸SO₂(C₁-C₄ alkyl), —S(O)ₙ(C₁-C₄ alkyl) wherein n is 0 to 2, —NR⁸C(O)C(O)NR⁸R⁶, —NR⁸C(O)C(O)R⁸, thiazolyl, imidazolyl, oxazolyl, pyrazolyl, triazolyl, tetrazolyl, and C₁-C₄ alkyl optionally substituted by 1 to 3 fluoro substituents;
each R⁶ and R⁷ is independently hydrogen or C₁-C₄ alkyl;
each R⁸ is independently R⁶ or —OR⁶; and,
each R⁹ is independently selected from cyano, R⁶, —OR⁶, —OC(O)R⁶, —C(O)OR⁶, —C(O)NR⁶R⁷, —NR⁶R⁷, —NR⁶R⁸, —SO₂NR⁶R⁷, and C₁-C₄ alkyl substituted by hydroxy; and
(b) compounds of the formula 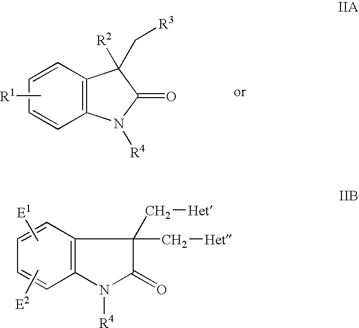
wherein R¹ is hydrogen, chloro, fluoro, bromo, iodo, cyano, hydroxy, nitro, trifluoromethyl, —NHR⁵, —NR⁵R⁵, R⁵, —OR ⁵or —S(O)ₘ—R⁵;
R² is —(CH₂)ₙ—Y or —OCOR⁵;
R³ is 4-, 3-, or 2-pyridyl, pyrimidyl, pyrazinyl, 2-fluoro-4-pyridyl or 3-fluoro4-pyridyl;
R⁴ is 1-adamantyl or 2-adamantyl;
Y is hydrogen, hydroxy, amino, cyano, —NHR₅, —NR⁵R⁵, —NHCOR⁵, —NHCO₂R⁵, halo, OR⁵, —S(O)ₘR⁵, —CO₂H, —CO₂R⁵, —CONR⁵R⁵, —CONHR⁵, —CONH₂, —COR⁵, —CH═CHCO₂R⁵, —OCOR⁵, phenyl, phenyl substituted with W, —C≡CCO₂R⁵, —CH═CHR or —C≡CR⁵;
each R⁵ is, independently, (C₁-C₄) straight or branched alkyl, phenyl or benzyl, wherein said phenyl and the phenyl moiety of said benzyl may optionally be substituted with halo, hydroxy, nitro, cyano, amino, (C₁-C₄) straight or branched alkyl, (C₁-C₄) straight or branched alkoxy, phenyl, benzyl, (C₁-C₄)alkylamino, di[(C₁-C₄)alkyl]amino, or —S(O)ₘ—(C₁-C₄) straight or branched alkyl;
each W is, independently, halo, R⁵, hydroxy, —OR⁵, nitro, amino, —NHR⁵, —NR⁵R⁵, cyano, or —S(O)ₘ—R⁵;
m is 0, 1 or 2;
n is 1 to 7;
p is 0 or 1;
E¹ and E² are selected, independently, from hydrogen, halo, (C₁-C₃)alkyl, hydroxy, (C₁-C₃)alkoxy, nitro, trifluoromethyl, cyano, amino, (C₁-C₃)alkylamino and di[(C₁-C₃)alkyl]amino;
and their pharmaceutically acceptable salts.
Het′ and Het″ are selected, independently, from 6 membered heterocyclic rings containing from one to four nitrogen atoms as part of the ring, optionally substituted with one substituent selected from (C₁-C₃)alkyl, halo, hydroxy, (C₁-C₃)alkoxy, amino, (C₁-C₃)alkylamino and di[(C₁-C₃)alkyl]amino; and
(c) compounds of the formula 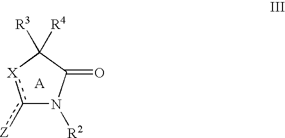
wherein both dotted lines represent optional double bonds;
Z is oxygen or sulfur when it is double bonded to ring A and Z is hydroxy, (C₁-C₁₀)alkyl-S—, (C₁-C₁₀)alkyl-SO—, (C₁-C₁₀)alkyl-SO₂—, adamant-2-yl-S—, naphthyl-S—, benzyl-S—, phenyl-C(═O)CH₂—S—, (C₁-C₆)alkyl-O—C(═O)—CH₂—S— or (H,H) (i.e., Z represents two hydrogen atoms, each of which is single bonded to the same carbon of ring A) when Z is single bonded to ring A, and wherein said naphthyl and phenyl and the phenyl moiety of said benzyl may optionally be substituted with from one to three substituents independently selected from (C₁-C₆)alkyl optionally substituted with from one to three fluorine atoms, (C₁-C₆)alkoxy optionally substituted with from one to three fluorine atoms, chloro, fluoro, bromo, iodo, amino, (C₁-C₆)alkylamino, [di-(C₁-C₆)alkyl]amino, cyano, nitro, (C₁-C₆)alkyl-SOₙ— wherein n is zero, one or two, —COOH, —COO(C₁-C₆)alkyl and —C(O)NH(C₁-C₆)alkyl;
X is NR¹ or CHR¹;
R¹ is hydrogen, (C₁-C₆)alkyl or (C₁-C₆)alkylphenyl when ring A is saturated (i.e., when ring A contains no double bonds) and R¹ is absent when ring A contains a double bond;
R² is selected from naphthyl, phenyl, (C₁-C₆)alkylphenyl, 1-adamantyl, 2-adamantyl, (C₁-C₈) straight or branched alkyl, (C₃-C₁₀) cycloalkyl and (C₈-C30)bicyclic or tricyclic alkyl; wherein said (C₃-C₁₀)cycloalkyl and said (C₈-C30)bicyclic or tricyclic alkyl may optionally be substituted with a hydroxy group; and wherein said adamantyl groups may optionally be substituted with from one to three substituents independently selected from (C₁-C₆)alkyl, halo and hydroxy; and
R³ and R⁴ are independently selected from benzyl, wherein the phenyl moiety of said benzyl may optionally be substituted with an amino or nitro group; hydrogen, phenyl, (N≡C)—(C₁-C₆)alkyl, (C₁-C₆)alkyl-O—C(═O)—(C₁-C₆)alkyl and Het-CH₂, wherein Het is selected from 2-, 3- or 4-pyridinyl, furyl, tetrahydrofuryl, pyrimidyl, pyrazinyl, pyrazolyl, isoxazolyl, thiophenyl and triazolyl;
with the proviso that (a) no more than one of the two dotted lines can represent a double bond in any one compound, (b) when Z is (H, H), X is CH₂, (c) when Z is oxygen or (H, H) and X is CHR¹, R¹ must be hydrogen, (d) when Z is sulfur and X is NR¹, R¹ must be hydrogen, and (e) one of R³ and R⁴ must be Het-CH₂; and
(d) the compound 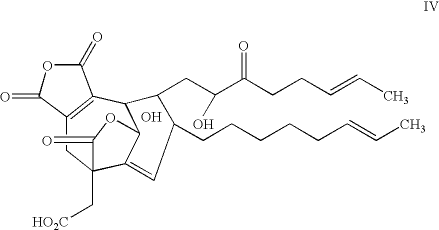
and the pharmaceutically acceptable salts of the foregoing compounds.

8. The composition of claim 7, wherein said hydroxymethylglutaryl coenzyme A reductase inhibitor is selected inhibitor is selected from the group consisting of atorvastatin, pravastatin, lovastatin, compactin, fluvastatin and simvastatin, and the pharmaceutically acceptable salts of the foregoing compounds.

9. A pharmaceutical composition comprising a Farnesyl transferase inhibitor and a hydroxymethylglutaryl coenzyme A reductase inhibitor and a pharmaceutically acceptable carrier, wherein said Farnesyl transferase inhibitor is selected from: (a) compounds of the formula 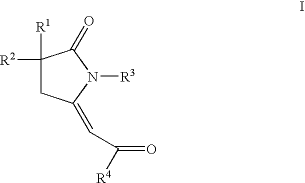
wherein R¹ and R² are independently selected from the group consisting of —(CH₂)ₚ(5-10 membered heterocycles), —(CH₂)ₚ(C₆-C₁₀ aryl), allyl, propargyl and C₁-C₆ alkyl wherein p is 0 to 3, said alkyl and the alkyl moieties of said R¹ and R² groups are optionally substituted by 1 to 3 R⁹ substituents, and the aryl and heterocyclic moieties of said R¹ and R² groups are optionally substituted by 1 to 3 substituents independently selected from halo and R⁹;
R³ is —(CH₂)ₘ(1- or 2-adamantyl), —(CH₂)ₘ(C₃-C₁₀ cycloalkyl), —(CH₂)ₘ(C₆-C₁₀ alkyl, 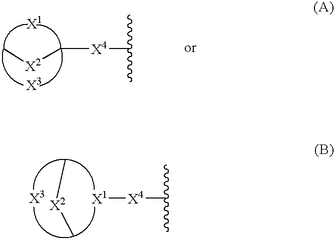
wherein m is 0 to 6, and said cycloalkyl and alkyl optionally contain 1 or 2 double or triple bonds;
X¹, X², and X³ are each independently C₁-C₇ alkylene optionally containing 1 or 2 double or triple bonds, X⁴ is a bond or C₁-C₇ alkylene optionally containing 1 or 2 double or triple bonds, and, in formula (B), the X⁴ moiety is attached to the X¹ moiety at any available carbon in the X¹ moiety;
R⁴is C₆-C₁₀ aryl, 5-10 membered heterocyclyl or C₁-C₆ alkyl wherein each of said R⁴ groups is optionally substituted by 1 to 3 R⁵ substituents;
each R⁵ is independently selected from the group consisting of halo, nitro, cyano, phenyl, —C(O)OR⁶, —SO₂NR⁶R⁷, —NR⁶R⁸, —C(O)R⁶, —OR⁶, —C(O)NR⁶R⁸, —OC(O)NR ⁶R⁸, —NR⁸C(O)NR⁸R⁶, —NR⁸C(O)R⁶, —NR⁸C(O)O(C₁-C₄ alkyl), —C(NR⁸)NR⁸R⁶, —C(NCN)NR⁸R⁶, —C(NCN)S(C₁-C₄ alkyl), —NR⁸C(NCN)S(C₁-C₄ alkyl), —NR C(NCN)NR⁸R⁶, —NR⁸SO₂(C₁-C₄ alkyl), —S(O)ₙ(C₁-C₄ alkyl) wherein n is 0 to 2, —NR⁸C(O)C(O)NR⁸R⁶, —NR⁸C(O)C(O)R⁸, thiazolyl, imidazolyl, oxazolyl, pyrazolyl, triazolyl, tetrazolyl, and C₁-C₄ alkyl optionally substituted by 1 to 3 fluoro substituents;
each R⁶ and R⁷ is independently hydrogen or C₁-C₄ alkyl;
each R⁸ is independently R⁶ or —OR⁶; and,
each R⁹ is independently selected from cyano, R⁶, —OR⁶, —OC(O)R⁶, —C(O)OR⁶, —C(O)NR⁶R⁷, —NR⁶R⁷, —NR⁶R⁸, —SO₂NR⁶R⁷, and C₁-C₄ alkyl substituted by hydroxy; and
(b) compounds of the formula 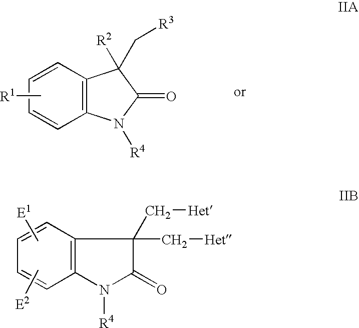
wherein R¹ is hydrogen, chloro, fluoro, bromo, iodo, cyano, hydroxy, nitro, trifluoromethyl, —NHR⁵, —NR⁵R⁵, R⁵, —OR⁵ or —S(O)ₘ—R⁵;
R² is —(CH₂)ₙ—Y or —OCOR⁵;
R³is 4-, 3-, or 2-pyridyl, pyrimidyl, pyrazinyl, 2-fluoro-4-pyridyl or 3-fluoro-4-pyridyl;
R⁴ is 1 -adamantyl or 2-adamantyl;
Y is hydrogen, hydroxy, amino, cyano, —NHR⁵, —NR⁵R⁵, —NHCOR⁵, —NHCO₂R⁵, halo, OR⁵, —S(O)ₘR⁵, —CO₂H, —CO₂R⁵, —CONR⁵R⁵, —CONHR⁵, —CONH₂, —COR⁵, —CH═CHCO₂R⁵, —OCOR⁵, phenyl, phenyl substituted with W, —C≡CCO₂R⁵, —CH═CHR⁵ or —C≡CR⁵,
each R⁵ is, independently, (C₁-C₄) straight or branched alkyl, phenyl or benzyl, wherein said phenyl and the phenyl moiety of said benzyl may optionally be substituted with halo, hydroxy, nitro, cyano, amino, (C₁-C₄) straight or branched alkyl, (C₁-C₄) straight or branched alkoxy, phenyl, benzyl, (C₁-C₄)alkylamino, di[(C₁-C₄)alkyl]amino, or —S(O)ₘ—(C₁-C₄) straight or branched alkyl;
each W is, independently, halo, R⁵, hydroxy, —OR⁵, nitro, amino, —NHR⁵, —NR⁵R⁵, cyano, or —S(O)ₘ—R⁵;
m is 0, 1or 2;
n is 1 to 7;
p is 0 or 1;
E¹ and E² are selected, independently, from hydrogen, halo, (C₁-C₃)alkyl, hydroxy, (C₁-C₃)alkoxy, nitro, trifluoromethyl, cyano, amino, (C₁-C₃)alkylamino and di[(C₁-C₃)alkyl]amino;
and their pharmaceutically acceptable salts.
Het′ and Het″ are selected, independently, from 6 membered heterocyclic rings containing from one to four nitrogen atoms as part of the ring, optionally substituted with one substituent selected from (C₁-C₃)alkyl, halo, hydroxy, (C₁-C₃)alkoxy, amino, (C₁-C₃)alkylamino and di[(C₁-C₃)alkyl]amino; and
(c) compounds of the formula 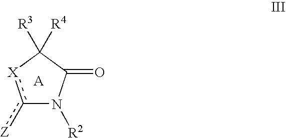
wherein both dotted lines represent optional double bonds;
Z is oxygen or sulfur when it is double bonded to ring A and Z is hydroxy, (C₁-C₁₀)alkyl-S—, (C₁-C₁₀)alkyl-SO—, (C₁-C₁₀)alkyl-SO₂—, adamant-2-yl-S—, naphthyl-S—, benzyl-S—, phenyl-C(═O)CH₂—S—, (C₁-C₆)alkyl-O—C(═O)—CH₂—S— or (H,H) (i.e., Z represents two hydrogen atoms, each of which is single bonded to the same carbon of ring A) when Z is single bonded to ring A, and wherein said naphthyl and phenyl and the phenyl moiety of said benzyl may optionally be substituted with from one to three substituents independently selected from (C₁-C₆)alkyl optionally substituted with from one to three fluorine atoms, (C₁-C₆)alkoxy optionally substituted with from one to three fluorine atoms, chloro, fluoro, bromo, iodo, amino, (C₁-C₆)alkylamino, [di-(C₁-C₆)alkyl]amino, cyano, nitro, (C₁-C₆)alkyl-SOₙ— wherein n is zero, one or two, —COOH, —COO(C₁-C₆)alkyl and —C(O)NH(C₁-C₆)alkyl;
X is NR¹ or CHR¹;
R¹ is hydrogen, (C₁-C₆)alkyl or (C₁-C₆)alkylphenyl when ring A is saturated (i.e., when ring A contains no double bonds) and R¹ is absent when ring A contains a double bond;
R² is selected from naphthyl, phenyl, (C₁-C₆)alkylphenyl, 1-adamantyl, 2-adamantyl, (C₁-C₈) straight or branched alkyl, (C₃-C₁₀) cycloalkyl and (C₈-C₃₀)bicyclic or tricyclic alkyl; wherein said (C₃-C₁₀)cycloalkyl and said (C₈-C₃₀)bicyclic or tricyclic alkyl may optionally be substituted with a hydroxy group; and wherein said adamantyl groups may optionally be substituted with from one to three substituents independently selected from (C₁-C₆)alkyl, halo and hydroxy; and
R³ and R⁴ are independently selected from benzyl, wherein the phenyl moiety of said benzyl may optionally be substituted with an amino or nitro group; hydrogen, phenyl, (N≡C)—(C₁-C₆)alkyl, (C₁-C₆)alkyl-O—C(═O)—(C₁-C₆)alkyl and Het-CH₂, wherein Het is selected from 2-, 3- or 4-pyridinyl, furyl, tetrahydrofuryl, pyrimidyl, pyrazinyl, pyrazolyl, isoxazolyl, thiophenyl and triazolyl;
with the proviso that (a) no more than one of the two dotted lines can represent a double bond in any one compound, (b) when Z is (H, H), X is CH₂, (c) when Z is oxygen or (H, H) and X is CHR¹, R¹ must be hydrogen, (d) when Z is sulfur and X is NR¹, R¹ must be hydrogen, and (e) one of R³ and R⁴ must be Het-CH₂; and
(d) the compound 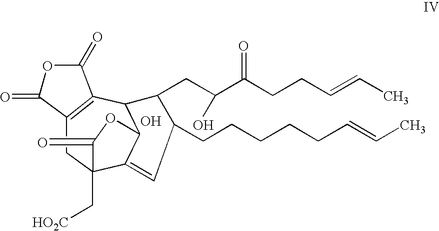
and the pharmaceutically acceptable salts of the foregoing compounds.

10. The pharmaceutical composition of claim 9, wherein said hydroxymethylglutaryl coenzyme A reductase inhibitor is selected inhibitor is selected from the group consisting of atorvastatin, pravastatin, lovastatin, compactin, fluvastatin and simvastatin, and the pharmaceutically acceptable salts of the foregoing compounds.

11. A method of treating cancer or benign proliferative disorder which comprises administering therapeutically effective amounts of a FTase inhibitor and a hydroxymethylglutaryl coenzyme A reductase inhibitor, wherein said Farnesyl transferase inhibitor is selected from: (a) compounds of the formula 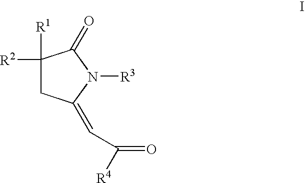
wherein R¹ and R² are independently selected from the group consisting of —(CH₂)ₚ(5-10 membered heterocycles), —(CH₂)ₚ(C₆-C₁₀ aryl), allyl, propargyl and Cₗ-C₈ alkyl wherein p is 0 to 3, said alkyl and the alkyl moieties of said R¹ and R² groups are optionally substituted by 1 to 3 R⁹ substituents, and the aryl and heterocyclic moieties of said R¹ and R² groups are optionally substituted by 1 to 3 substituents independently selected from halo and R⁹;
R³ is —(CH₂)ₘ(1- or 2-adamantyl), —(CH₂)ₘ(C₃-C₁₀ cycloalkyl), —(CH₂)ₘ(C₆-C₁₀ alkyl, 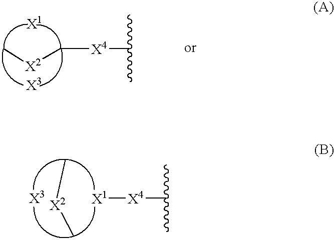
wherein m is 0 to 6, and said cycloalkyl and alkyl optionally contain 1 or 2 double or triple bonds;
X¹, X², and X³ are each independently C₁-C₇ alkylene optionally containing 1 or 2 double or triple bonds, X⁴ is a bond or C₁-C₇ alkylene optionally containing 1 or 2 double or triple bonds, and, in formula (B), the X⁴ moiety is attached to the X¹ moiety at any available carbon in the X¹ moiety;
R⁴ is C₆-C₁₀ aryl, 5-10 membered heterocyclyl or C₁-C₆ alkyl wherein each of said R⁴ groups is optionally substituted by 1 to 3 R⁵ substituents;
each R⁵ is independently selected from the group consisting of halo, nitro, cyano, phenyl, —C(O)OR⁶, —SO₂NR⁶R⁷, —NR⁶R⁸, —C(O)R⁶, —OR⁶, —C(O)NR⁶R⁸, —OC(O)NR⁶R⁸, —NR⁸C(O)NR⁸R⁶, —NR⁸C(O)R⁶, —NR⁸C(O)O(C₁-C₄ alkyl), —C(NR⁸)NR⁸R⁶, —C(NCN)NR⁸R⁶, —C(NCN)S(C₁-C₄ alkyl), —NR⁸C(NCN)S(C₁-C₄ alkyl), —NR⁸C (NCN)NR⁸R⁶, —NR⁸SO₂(C₁-C₄ alkyl, —S(O)ₙ(C₁-C₄ alkyl) wherein n is 0 to 2, —NR⁸C(O)C(O)NR⁸R⁶, —NR⁸C(O)C(O)R⁸, thiazolyl, imidazolyl, oxazolyl, pyrazolyl, triazolyl, tetrazolyl, and C₁-C₄ alkyl optionally substituted by 1 to 3 fluoro substituents;
each R⁶ and R⁷ is independently hydrogen or C₁-C₄ alkyl;
each R⁸ is independently R⁶ or —OR⁶; and,
each R⁹ is independently selected from cyano, R⁶, —OR⁶, —OC(O)R⁶, —C(O)OR⁶, —C(O)NR⁶R⁷, —NR⁶R⁷, —NR⁶R⁸, —SO₂NR⁶R⁷, and C₁-C₄ alkyl substituted by hydroxy; and
(b) compounds of the formula 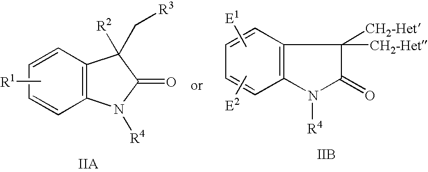
wherein R¹ is hydrogen, chloro, fluoro, bromo, iodo, cyano, hydroxy, nitro, trifluoromethyl, —NHR⁵, —NR⁵R⁵, R⁵, —OR⁵ or —S(O)ₘ—R⁵;
R² is —(CH₂)ₙ—Y or —OCOR⁵;
R³ is 4-, 3-, or 2-pyridyl, pyrimidyl, pyrazinyl, 2-fluoro-4-pyridyl or 3-fluoro-4-pyridyl;
R⁴ is 1-adamantyl or 2-adamantyl;
Y is hydrogen, hydroxy, amino, cyano, —NHR⁵, —NR⁵R⁵, —NHCO⁵, —NHCO₂R⁵, halo, OR⁵, —S(O)ₘR⁵, —CO₂H, —CO₂R⁵, —CONR⁵R⁵, —CONHR⁵, —CONH₂, —COR⁵, —CH═CHCO₂R⁵, —OCOR⁵, phenyl, phenyl substituted with W, —C≡CCO₂R⁵, —CH═CHR⁵, or —C≡CR⁵,
each R⁵ is, independently, (C₁-C₄) straight or branched alkyl, phenyl or benzyl, wherein said phenyl and the phenyl moiety of said benzyl may optionally be substituted with halo, hydroxy, nitro, cyano, amino, (C₁-C₄) straight or branched alkyl, (C₁-C₄) straight or branched alkoxy, phenyl, benzyl, (C₁-C₄)alkylamino, di[(C₁-C₄)alkyl]amino, or —S(O)ₘ—(C₁-C₄) straight or branched alkyl;
each W is, independently, halo, R⁵, hydroxy, —OR⁵, nitro, amino, —NHR⁵, —NR⁵R⁵, cyano, or —S(O)ₘ—R⁵;
m is 0, 1 or 2;
n is 1 to 7;
p is 0 or 1;
E¹ and E² are selected, independently, from hydrogen, halo, (C₁-C₃)alkyl, hydroxy, (C₁-C₃)alkoxy, nitro, trifluoromethyl, cyano, amino, (C₁-C₃)alkylamino and di[(C₁-C₃)alkyl]amino;
and their pharmaceutically acceptable salts.
Het′ and Het′ are selected, independently, from 6 membered heterocyclic rings containing from one to four nitrogen atoms as part of the ring, optionally substituted with one substituent selected from (C₁-C₃)alkyl, halo, hydroxy, (C₁-C₃)alkoxy, amino, (C₁-C₃)alkylamino and di[(C₁-C₃)alkyl]amino; and
(c) compounds of the formula 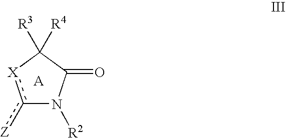
wherein both dotted lines represent optional double bonds;
Z is oxygen or sulfur when it is double bonded to ring A and Z is hydroxy, (C₁-C₁₀)alkyl-S—, (C₁-C₁₀)alkyl-SO—, (C₁-C₁₀)alkyl-SO₂—, adamant-2-yl-S—, naphthyl-S—, benzyl-S—, phenyl-C(═O)CH₂-S—, (C₁-C₆)alkyl-O—C(═O)—CH₂—S— or (H,H) (i.e., Z represents two hydrogen atoms, each of which is single bonded to the same carbon of ring A) when Z is single bonded to ring A, and wherein said naphthyl and phenyl and the phenyl moiety of said benzyl may optionally be substituted with from one to three substituents independently selected from (C₁-C₆)alkyl optionally substituted with from one to three fluorine atoms, (C₁-C₆)alkoxy optionally substituted with from one to three fluorine atoms, chloro, fluoro, bromo, iodo, amino, (C₁-C₆)alkylamino, [di-(C₁-C₆)alkyl]amino, cyano, nitro, (C₁-C₆)alkyl-SOₙ— wherein n is zero, one or two, —COOH, —COO(C₁-C₆)alkyl and —C(O)NH(C₁-C₆)alkyl;
X is NR¹ or CHR¹;
R¹ is hydrogen, (C₁-C₆)alkyl or (C₁-C₆)alkylphenyl when ring A is saturated (i.e., when ring A contains no double bonds) and R¹ is absent when ring A contains a double bond;
R² is selected from naphthyl, phenyl, (C₁-C₆)alkylphenyl, 1-adamantyl, 2-adamantyl, (C₁-C₈) straight or branched alkyl, (C₃-C₁₀) cycloalkyl and (C₈-C₃₀)bicyclic or tricyclic alkyl; wherein said (C₃-C₁₀)cycloalkyl and said (C₈-C₃₀)bicyclic or tricyclic alkyl may optionally be substituted with a hydroxy group; and wherein said adamantyl groups may optionally be substituted with from one to three substituents independently selected from (C₁-C₆)alkyl, halo and hydroxy; and
R³ and R⁴ are independently selected from benzyl, wherein the phenyl moiety of said benzyl may optionally be substituted with an amino or nitro group; hydrogen, phenyl, (N≡C)—(C₁-C₆)alkyl, (C₁-C₆)alkyl-O—C(═O)—(C₁-C₆)alkyl and Het-CH₂, wherein Het is selected from 2-, 3- or 4-pyridinyl, furyl, tetrahydrofuryl, pyrimidyl, pyrazinyl, pyrazolyl, isoxazolyl, thiophenyl and triazolyl;
with the proviso that (a) no more than one of the two dotted lines can represent a double bond in any one compound, (b) when Z is (H, H), X is CH₂, (c) when Z is oxygen or (H, H) and X is CHR¹, R¹ must be hydrogen, (d) when Z is sulfur and X is NR¹, R¹ must be hydrogen, and (e) one of R³ and R⁴ must be Het-CH₂; and
(d) the compound 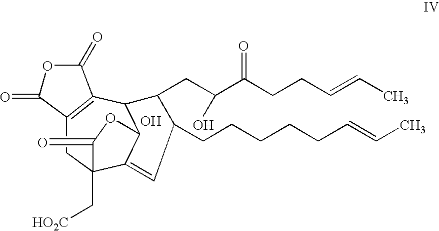
and the pharmaceutically acceptable salts of the foregoing compounds.

12. The method of claim 11, wherein said hydroxymethylglutaryl coenzyme A reductase inhibitor is selected inhibitor is selected from the group consisting of atorvastatin, pravastatin, lovastatin, compactin, fluvastatin and simvastatin, and the pharmaceutically acceptable salts of the foregoing compounds.
